(19) United States
(12) Patent Application Publication
(10) Pub. No.: US 2007/0193874 A1
(43) Pub. Date: Aug. 23, 2007

(54) Method and device for improved fermentation process

(76) Inventors: Kayyani C. Adiga, Macon, GA (US); Rajani Adiga, Macon, GA (US)

Correspondence Address: BRIAN D. BELLAMY, P.O. BOX 1997, THOMASVILLE, GA, 31799-1997, US

(21) Appl. No.: 11/674,948

(22) Filed: Feb. 14, 2007

Related U.S. Application Data
(60) Provisional application No. 60/766,847, filed on Feb. 14, 2006.

(51) Int. Cl. C12P 7/06; B01D 3/00

(52) U.S. Cl. 203/99; 435/161

(57) ABSTRACT

An improved method and device is accomplished for ethanol production using an in-line extraction of ethanol by ultrasonic atomization, thereby removing the effect of the ethanol inhibition factor that adversely affects the rate and yield. The in-line removal of ethanol as it is formed increases the fermentation rate, improves the yield and uses 20-25% of the energy required as compared to thermal distillation processes currently used. The improved method makes sure that ultrasonic vibration does not deactivate enzymes to any significant level. The elimination of the effect of the ethanol inhibition factor allows for a continuous fermentation process as opposed to the costly, time-consuming repeated batch processes.

### PRIORITY CLAIM

The present application claims benefit of the U.S. provisional patent application No. 60/766,847 filed on Feb. 14, 2006.

### BACKGROUND OF INVENTION

This invention relates to the in-line extraction and removal of ethanol formed in a fermentation process. In particular, ethanol is removed from a reaction mixture using an ultrasonic atomization process without subjecting the mixture of fermentable sugar and fermentation organism to thermal distillation or exceeding the organism's ethanol tolerance level, thereby improving the term of anaerobic respiration by the organism.

In a fermentation process, yeast or other organisms are added to a mash to ferment the sugars. In a well-known process, yeast, cut off from oxygen, will ferment a starchy grain or vegetable such as wheat, corn, potatoes or rye. Sugar cane is also a common feed stock, and cellulosic materials, such as wood, are being considered for conversion to fermentable sugars using enzymes or other suitable processes. Fermentation breaks down the sugar molecules into ethanol and carbon dioxide.

The fermentation process can be either a batch process or what is presently considered a continuous process. In the current batch process, the ethanol is obtained by distillation after terminating the fermentation process. In existing continuous fermentation processes, fermentation reactors are piped together in series. These processes are really a plurality of batch feed processes as opposed to a continuous in-feed of mash.

The present batch feed continuous process accomplishes, with certain drawbacks, continuous removal of ethanol and the spent product from the reactor. By the time the mash enters the final fermentation reactor, the reactant material or sugar has reacted to the extent possible, depending on the ethanol inhibition factor or ethanol tolerance limit (ETL). ETL is the concentration of ethanol (% v/v) in the reactor above which yeast will be inactive and the rate of ethanol production will fall. In batch fermentation, the mash stays in one fermentation tank for approximately 2 days to allow complete fermentation. The reaction does not complete to its fullest extent because ethanol inhibits the reaction rate as indicated above.

In the distillation process, the reacted mixture is subjected to multicolumn distillation, which removes the alcohol utilizing the differences in the boiling point of ethanol and water. This liquid condensate is passed to successive distillation columns in the series, where the process is repeated. By the time the product reaches the final distillation column, it is 96% ethanol, or about 190 proof. The residue from distillation, called stillage, is pumped from the bottom of these distillation columns. The 190 proof ethanol is then passed through a molecular sieve, which removes remaining water that was not eliminated in distillation. Following dehydration, the ethanol is 200 proof and is referred to as anhydrous ethanol, which means ethanol without water.

Understanding the effect of ethanol on yeast or organism cells and enzymes that may be used during the process of creating the reactant material is very important for the biofuel production industry. During biofuel fermentation, the rising concentration of ethanol tends to have an inhibitory effect on fermentation via complex effects on cells. The overall rate of fermentation is influenced by osmotic pressure, ethanol concentration of the medium and sugar tolerance of the yeast strain employed. During the early stages of fermentation, intracellular accumulation of ethanol is observed in synthetic media and wort. When fermentation has reached 5%-7% alcohol concentration, fermentation cannot be re-started with this yeast because the ethanol tolerance is exceeded. Very few types of yeasts can re-start fermentation over 7% alcohol. The need to prevent the effect of the ethanol inhibition on organisms not only applies to sugar fermentation by yeast but is applicable to some degree with respect to all fermentation using organisms. Moreover, every known organism has a certain level of ethanol at which the organism can not survive or remain active. Additionally, enzymatic processes used in creation of fermentable reactant material may be heat sensitive.

Process technology improvements are needed foremost to save energy in distillation and improve production of ethanol as an alternative to petroleum fuels. Removing or eliminating the effect of the ethanol-inhibiting factor will help to increase the rate and the yield of ethanol and prevent the premature aborting of the fermentation reaction. Further, removing or eliminating the effect of the ethanol inhibition factor will permit the use of a more aptly described continuous process as opposed to aborting the fermentation process and running in batches.

### SUMMARY OF THE INVENTION

This invention provides an in-line process for the extraction and removal of ethanol formed in a fermentation process. In particular, ethanol is removed from a reaction mixture of fermentable reactant material, such as sugar, and other organism, such as yeast, using an ultrasonic atomization process. While yeasts are commonly known as the preferred fermentation organisms, alternative organisms are contemplated, and enzymatic processes involved in the production of ethanol may also be improved by the methods of the invention. The reaction mixture is not subjected to thermal distillation. Thereby, the activity of the yeast is not disturbed by heating of the mixture. Furthermore, the present invention maintains the fermentation reaction continuously by extracting ethanol from the reaction mixture in-line, without affecting the yeast activity significantly. The continuous in-line extraction of ethanol prevents exceeding ethanol tolerance.

The present invention utilizes ultrasonic atomization of the reaction mixture to separate more volatile ethanol from the mixture via cavitations and/or capillary breakup mechanisms. Ultrasonic atomization of the reaction mixture results from compression and rarefaction waves formed within the liquid. These compression and rarefaction waves create enormous energy and pressure within very short time scales. The rate and efficiency of atomization directly relates to the liquid vapor pressure boiling point. When water and ethanol are mixed and atomized, ethanol, being the more volatile component, will be richer in the mist-phase product. The richer mist-phase product is separable from the reaction mixture as it is formed, providing a product higher in ethanol content and removing ethanol from the reaction mixture. The separation ability and the energy efficiency of this atomization separation process are excellent compared with distillation.

It is also possible to increase efficiency further by widening the gap between the boiling point of ethanol and water. The boiling point of the water can be increased by forming a micro-emulsion in the presence of a surfactant. Increasing the boiling point of water will further help in separating ethanol and water by an ultrasonic misting process. It has been shown that the boiling point of water can be increased as much as 10-20° C., depending on the chain-length of oil added to water to make the micro-emulsion system.

A mist, which is rich in ethanol, is collected by electrostatic means and/or a de-misting matrix, wherein alcohol is recovered. As discussed above, the ultrasonic atomization separates and removes a major portion of ethanol produced in the reaction mixture. Otherwise, ever increasing ethanol concentrations in the reaction mixture would become an inhibiting factor for the function of yeast and would slow down the fermentation process and require batch processing.

An objective of this invention is to devise a continuous process of ethanol production using in-line removal of ethanol by not subjecting the reaction mixture to heat or thermal distillation, which kills the organism used in fermentation. Using these novel innovations, the process can now be carried out uninterrupted by continuously feeding reactants (such as sugar+water) to fermentation organisms, removing alcohol, and periodically removing the “spent” material. Successfully creating an environment for the complete fermentation and conversion of the substrate provides a process that supports the industry's goal of increasing yields.

A second objective is to provide a non-thermal process technology to reduce the energy consumed for separating ethanol from water and a reaction mixture. The energy for distillation based on the latent heat of vaporization of ethanol is 884 kJ/kg, without considering the heat transfer limitations (efficiency factor<1.0) in distillation. The atomization process of the current invention requires less than 215 kJ/kg of energy for distillation based on the energy required to atomize ethanol liquid into 1-5 micron droplets. This is only about 25% of the energy required for thermal distillation, which is a big saving in view of the present oil dependence and energy shortage. While modern distillation processes have heat recovery systems to recover latent energy input, thermal distillation still requires significant sensible enthalpy input. This energy is input in order to heat the entire mixture to its boiling point. Significant reduction of the energy requirements for conversion of the mash to ethanol will positively influence the industry's production cost per gallon and significantly increase the net margin of fuel energy produced opposed to the energy used in production.

In addition to the positive energy efficiency provided, the present invention will provide a method for using well-characterized enzymes that are thermally intolerant and normally unavailable. Thus, by providing a non-thermal process, options for selection and engineering of enzymes and genetically engineered yeasts will increase and provide still further improvements in the production of ethanol.

Another objective is to use an ultrasonic atomization process to remove the ethanol produced by fermentation organism so that the ethanol concentration will not rise above a certain concentration at which it will kill or deactivate fermentation organism and reduce the rate of production. This “ethanol inhibition factor” is specific to each organism and is the critical concentration above which the ethanol becomes lethal to organisms. During the fermentation process, when the alcohol concentration reaches the ethanol tolerance level (ETL) of approximately 5%-15% by volume, the yeast activity significantly decreases, resulting in inhibited fermentation. This ethanol-inhibiting characteristic is currently a factor limiting and effecting production rate and yield of ethanol from fermentation. This ethanol tolerance factor for the process is still very low even for fermentation of cellulosic materials (5-6% (v/v). Therefore, the present invention is very important in view of the current interest in using cellulosic materials for production of ethanol.

The present invention provides a method and device that may be used in one objective of the invention to maintain the alcohol concentration below the ETL level. The system provides in-line atomization and extraction of alcohol while fermentation continues unabated. By maintaining an ethanol concentration level below the ETL, the fermentation process will continue until all available components are converted to alcohol with a high rate. The removal of ethanol “on-the-fly” cannot be done by thermal distillation because the high temperature will kill the commonly used organisms such as yeast. This approach increases the production rate, improves gallon/bushel yields, and reduces energy requirements for thermal distillation and overall production.

Yet another objective is to provide a continuous process technology for fermentation that increases rate and yield as opposed to batch-by-batch processes as usually carried out in order to overcome the ethanol inhibition factor. Usually, the entire batch is stopped and terminated because of inhibition by ethanol and then started again.

Another objective is to provide a process that does not kill or deactivate the microorganism or fungi involved in the fermentation process to a significant level. Previously, ultrasonic vibrations have been known to kill or deactivate microorganisms such as yeast. Thus, it is desirable to remove alcohol by ultrasonic methods while not significantly affecting yeast activity.

Yet another objective is to use a minimum of carrier gas (air) to carry a mist generated by an ultrasonic mist extraction device.

Another objective is to extract and transport extremely fine mist with extremely low momentum so that an electrostatic or any other de-misting, mist collecting and coalescing process can be used to recover ethanol from the misting process.

### BRIEF DESCRIPTION OF THE DRAWINGS

FIG. 1 is a graph comparing the effects of alcohol production on the fermentation process using a continuous process in comparison to a batch process.

FIG. 2 is a schematic plan view of an embodiment of the invention for continuous fermentation by in-line removal of ethanol by a non-thermal process.

FIG. 3 is a graph showing the separation of ethanol and water from an ethanol and water mixture using a non-thermal distillation method of the invention.

FIG. 4 is a schematic plan view of an embodiment of the invention illustrating the separation of ultrasonic vibrations used for atomization from active fermentation area.

### DETAILED DESCRIPTION

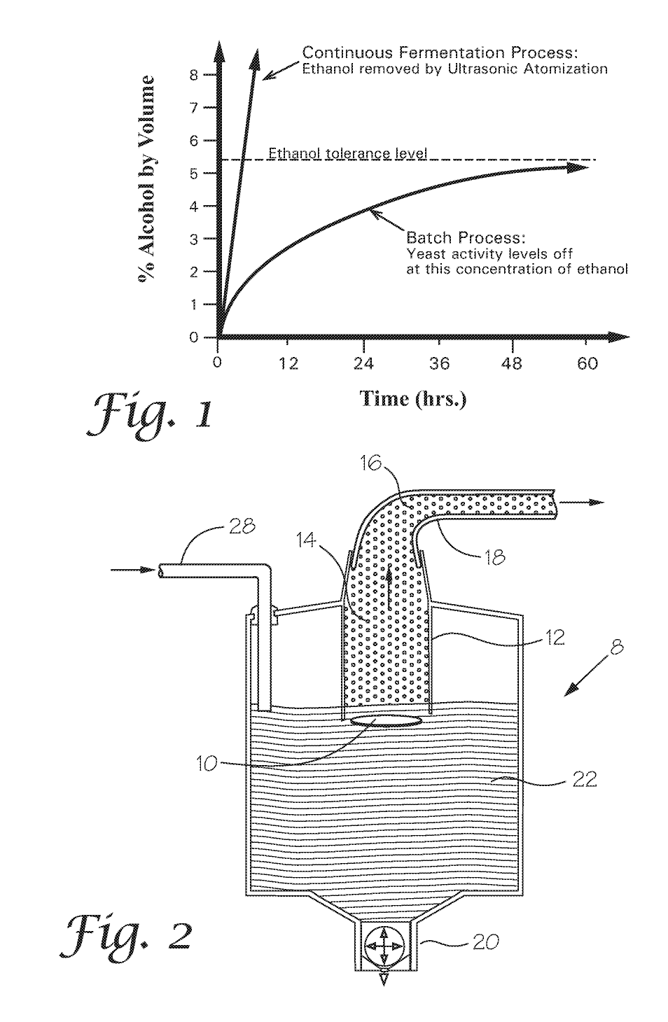

FIG. 1 shows a schematic graph of an exemplary and comparative reaction displaying progress in relation to time for a typical sugar-yeast fermentation process. As the ethanol concentration builds up, the rate starts slowing down; finally, the reaction practically terminates. The fermentation microorganisms above this stage are no longer active. The specific concentration of ethanol that slows and terminates the reaction process depends on the fermentation mixture and the nature of the fermentation organism used. The terminating ethanol concentration could be as low as 5-6% (v/v) for cellulosic materials using modern enzymes in the conversion to reactant material and fermentation process. Roughly, 12-20% is the limit for a sugar and yeast system, but higher levels are also reported. Certain strains of yeast used for fermentation of cellulosic material have a very low tolerance limit of about 5%. Irrespective of the specific concentration level, the very presence of such an inhibition factor calls for a batch process, since the reaction effectively stops at this point. If the effect of this inhibition factor is eliminated by extracting alcohol as it is formed, then the process could go to completion.

However, thermal distillation cannot be used for in-line continuous alcohol extraction because fermentation organisms will not survive the application of heat. As for ultrasonic atomization, the ultrasonic vibration generally kills the fermentation organism depending on frequency, amplitude, and energy of the vibrations. Therefore, a method is provided by the invention that minimizes killing the fermentation organism or restricts the atomizing pressure waves to a selected region so that the rest of the reaction mixture is free from pressure waves. The fermentation organism outside this selected region will continue to react with the reactant and produce alcohol. A reasonably high level of organism activity is maintained, while extracting ethanol continuously.

Referring to FIG. 2, the present process of removing ethanol from the fermentation mixture uses a fermentation reactor 8. A provision of reaction mixture is supplied to the fermentation reactor for the extraction of ethanol using an in-line atomization process. The reaction mixture comprises a reactant and fermentation organism. The reactant may be sugar and water or any other biomass product and process that may be converted into an appropriate sugar or reactant matter for fermentation. For instance, the reactant may include an alcohol convertible biomass.

The fermentation reactor 8 includes a high throughput ultrasonic atomization device such as the atomization disk 10 and a high throughput mist extracting device 12. The fermentation reactor provides for a portion or selected region of the reaction mixture to be subjected to atomizing pressure waves. A wide range of frequencies of pressure waves can be used to generate the high throughput mist 14, including a frequency of an ultrasonic vibrator of about 2.4 MHZ. The mist may be collected by force, such as by electrostatic voltage, to create an ethanol-rich mist outlet stream 16. A sufficient force producing mechanism should be used to successfully collect the mist and prevent waste. With an electrostatic voltage a range of −1,000 V to −10,000V would generally suffice.

Ethanol is removed on-line and inhibiting ethanol concentration levels at ambient temperature are prevented. The atomization process may involve ambient temperature atomization of liquid without using pressure nozzles. Ethanol is separated by collecting the atomized droplets from the outlet stream 16 coming out of ethanol traps 18 installed at the top of the fermentation reactor 8. As seen in FIG. 2, the trap 18 may be installed at the top of the reactor. Multiple traps may be installed depending on the size of the fermentation reactor. The removal process may be intermittent to match the demand for removal of inhibiting ethanol levels. Since the fermentation rate is usually slow compared to the atomization/removal rate, the process of on-line removal has to be suitably implemented into the process loop.

The spent material or sludge created by the fermentation process may be gravity dropped from the base with a fluid lock system 20 intermittently in order to keep the process continuous as shown at the base of the reactor in FIG. 2. Other methods of removal may be devised by those skilled in the art but are not specifically detailed here. Continuous removal of spent matter will generally be advantageous in accordance with the present invention to support and promote the ongoing fermentation process.

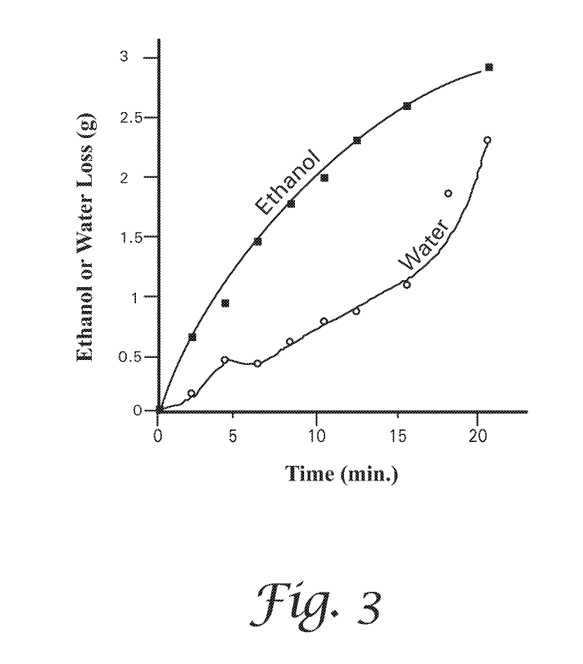

The ethanol separation process described in the invention provides a non-thermal distillation process using fine-scale atomization. FIG. 3 shows the results of non-thermal distillation of a mixture of ethanol and water. The graph shows that as a function of time, the atomized spray coming out of the reactor is richer in ethanol as compared to water. Thereby, this process in the fermentation reactor will remove ethanol and, when removal is continuous, the process can prevent ethanol concentration levels that would inhibit the rate of fermentation.

Using the non-thermal distillation process with fine-scale atomization, no heat or energy addition to the reactant mixture is necessary to separate the ethanol from the fermenting mass. Therefore, the active fermentation organisms are not damaged or killed by the addition of heat, as in conventional thermal distillation used in batch processes. Also, fungal amylase enzymes and other enzymes used in ethanol production or breaking down starches into sugars may benefit from elimination of heat used in thermal distillation. Instead, the ethanol separation is carried out in-line by atomization without terminating the fermentation process with the addition of heat. After separation of the ethanol rich mist to form an ethanol rich product, the product may be dehydrated further to produce anhydrous ethanol. A method to replace the dehydration process in typical thermal distillation would include passing the ethanol rich product through a molecular sieve, whereby remaining water in a 95% or like ethanol product is removed. Following complete removal of water and dehydration, the ethanol is 100% pure of 200 proof and referred to as anhydrous ethanol.

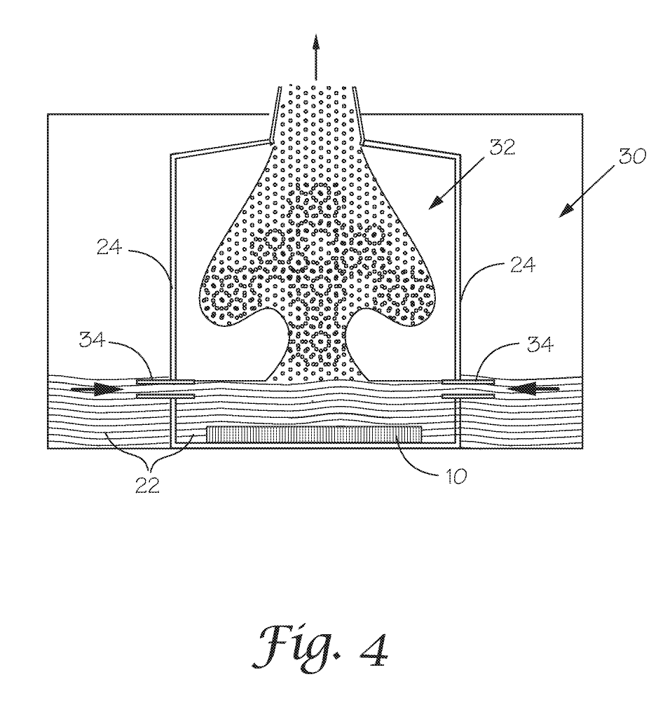

As shown in FIG. 4, the reaction between a reactant, such as sugar, and added fermentation organism, such as yeast or the like, takes place in the reaction mixture 22. To avoid the known detrimental effect of ultrasonic waves on the active organism, the present invention provides for the physical or sonic separation of a misting area from a larger fermentation area. Thus, a fermentation area is provided that is separated from the atomizing pressure waves. In one embodiment the vibration source is separated by a thin plastic wall 24 separating the disk 10 from the rest of the reaction mixture 22 of the fermentation area as shown in FIG. 4. A variety of separation methods may be employed such as physical barriers as shown by the wall 24 or separate feed lines as shown by the in-feed tube 28 in FIG. 2. The barrier 24 in FIG. 4 creates separate outer and inner chambers 30 and 32 containing the reaction mixture. Regions of the reaction mixture are physically separated by providing and an outer reaction mixture in the outer chamber 30 and an inner reaction mixture in the inner chamber 32. A connection 34 between the outer reaction mixture and the inner reaction mixture provides for a constant supply of reaction mixture to provide a selected region of the reaction mixture to the atomizing area.

Alternatively, the in-feed 28 of reaction mixture in FIG. 2 provides a flow of reaction material 22 through a conduit pipe from a spatially separated fermentation area to the atomizing area as needed. The flow of reaction material may be continuous or controlled and intermittent, whereby a portion of reaction material is introduced to the atomizing area to provide a selection region of the reaction material for atomizing and production of the ethanol rich mist 14. The spatial separation of the primary fermentation area from the atomizing area protects the bulk of the reactant material from the ultrasonic waves of the atomizing device. Therefore, the reaction is continuous and not significantly effected by the deleterious effects of the atomizer on the reactant material.

In another alternative to the schematic illustration of FIG. 2, the in-feed 28 may provide a supply of reactant separate from a supply of fermentation organism into the fermentation reactor. As the reactant and fermentation organisms are combined in the reactor, a reactant mixture 22 for fermentation is formed. The reaction mixture ferments to produce ethanol, and then an ethanol rich mist is generated from the reaction mixture by atomization. The ethanol is separated and removed from the reaction mixture by a suitable collection means, as the separate materials are fed into the fermentation reactor as needed for the continuing production of ethanol. Likewise, spent sludge from the fermentation reactor may be removed continuously to promote the ongoing reaction and production of ethanol.

It will be obvious to those skilled in the arts that substitutions and equivalents will exist for the elements of embodiments illustrated above.

We claim:

1. A method to extract and remove ethanol formed in a fermentation process including the steps of:
a) providing a reaction mixture of a reactant and a fermentation organism;
b) using atomizing pressure waves to generate an ethanol rich mist from the reaction mixture;
c) restricting the atomizing pressure waves to a selected region of the reaction mixture; and
d) separating and removing ethanol from the reaction mixture by collecting the mist.

2. A method to extract and remove ethanol formed in a fermentation process as in claim 1 in which the atomizing pressure waves used to generate an ethanol rich mist are provided by an ultrasonic atomizing disk.

3. A method to extract and remove ethanol formed in a fermentation process as in claim 1 in which the step of restricting the atomizing pressure waves to a selected region of the reaction mixture includes providing the reaction mixture to the atomizing pressure waves from a fermentation area that is separated from the atomizing pressure waves.

4. A method to extract and remove ethanol formed in a fermentation process as in claim 3 in which the fermentation area is physically separated from the atomizing pressure waves.

5. A method to extract and remove ethanol formed in a fermentation process as in claim 4 in which the fermentation area is physically separated by providing and an outer reaction mixture, an inner reaction mixture and a connection between the outer reaction mixture and the inner reaction mixture.

6. A method to extract and remove ethanol formed in a fermentation process as in claim 2 including the additional step of separating the reaction mixture from the ultrasonic atomizing disk using a diaphragm.

7. A method to extract and remove ethanol formed in a fermentation process as in claim 3 in which the fermentation area is spatially separated from the atomizing pressure waves.

8. A method to extract and remove ethanol formed in a fermentation process as in claim 7 in which the fermentation area is spatially separated from the atomizing pressure waves by feeding the reaction mixture from the fermentation area through a conduit pipe continuously or intermittently.

9. A method to extract and remove ethanol formed in a fermentation process as in claim 1 in which the step of restricting the atomizing pressure waves to a selected region of the reaction mixture prevents the atomizing pressure waves from killing or deactivating a quantity of the fermentation organism sufficient to adversely affect the fermentation rate.

10. A method to extract and remove ethanol formed in a fermentation process as in claim 1 in which the step of separating and removing ethanol from the reaction mixture by collecting the mist is done continuously.

11. A method to extract and remove ethanol formed in a fermentation process as in claim 1 including the step of controlling the ethanol concentration of the reaction mixture by in-line removal of ethanol during the fermentation process.

12. A method to extract and remove ethanol formed in a fermentation process as in claim 1 in which the fermentation organism is yeast, genetically engineered yeast, or bacteria.

13. A method to extract and remove ethanol formed in a fermentation process as in claim 1 in which the fermentation organism is an enzyme that converts a biomass into alcohol.

14. A method to extract and remove ethanol formed in a fermentation process as in claim 1 in which the reactant is an alcohol convertible biomass.

15. A method to extract and remove ethanol formed in a fermentation process as in claim 1 in which the reactant is sugar.

16. A method to extract and remove ethanol formed in a fermentation process as in claim 1 in which the separating and removing ethanol from the reaction mixture by collecting the atomized mist includes using at least one ethanol trap or electrostatic voltage.

17. A method to extract and remove ethanol formed in a fermentation process as in claim 1 including the step of providing the fermentation process in a batch.

18. A method to extract and remove ethanol formed in a fermentation process including the steps of:
a) separately feeding a reactant and a fermentation organism into a reactor to form a reactant mixture for fermentation;
b) generating an ethanol rich mist from the reaction mixture;
c) separating and removing ethanol from the reaction mixture by collecting the atomized mist.

19. A method to extract and remove ethanol formed in a fermentation process as in claim 18 in which the step of separately feeding the reactant and the fermentation organism into the reactor to form the reactant mixture for fermentation is continuous and further includes the step of removing a spent sludge created by the fermentation process from the reactor as needed for continuous fermentation.

20. A method to extract and remove ethanol formed in a fermentation process as in claim 19 in which the spent sludge created by the fermentation process is removed by gravity dropping the spent sludge from the reactor intermittently via a fluid lock system.

21. A method to extract and remove ethanol formed in a fermentation process including the steps of:
a) providing a fermentation reaction including an oil-in-water micro-emulsion, wherein the water's boiling point temperature is increased;
b) using atomizing pressure waves to generate an ethanol rich mist from the fermentation reaction;
c) separating and removing ethanol from the fermentation reaction by collecting the mist.

22. A method to extract and remove ethanol formed in a fermentation process as in claim 1 including the additional step of making a product of the ethanol rich mist completely anhydrous.

23. A method to extract and remove ethanol formed in a fermentation process as in claim 22 in which the step of making the product of the ethanol rich mist completely anhydrous includes removing remaining water from the product of the ethanol rich mist by passing the product through a molecular sieve.
